(19) United States
(12) Patent Application Publication
(10) Pub. No.: US 2022/0027686 A1
(43) Pub. Date: Jan. 27, 2022

(54) DATA PROCESSING APPARATUS, DATA PROCESSING METHOD, AND PROGRAM

(71) Applicant: Nippon Telegraph and Telephone Corporation, Tokyo (JP)

(72) Inventors: Akihiro Chiba, Tokyo (JP); Shozo Azuma, Tokyo (JP); Kazuhiro Yoshida, Tokyo (JP); Hisashi Kurasawa, Tokyo (JP); Naoki Asanoma, Tokyo (JP); Kana Eguchi, Tokyo (JP); Tsutomu Yabuuchi, Tokyo (JP)

(21) Appl. No.: 17/276,767

(22) PCT Filed: Sep. 17, 2019
(86) PCT No.: PCT/JP2019/036262

§ 371 Date: Mar. 16, 2021

(51) Int. Cl. G06K 9/62; G06F 17/17; G06F 17/18

(52) U.S. Cl. CPC G06K 9/6298; G06K 9/6262; G06F 17/18; G06F 17/175; G06K 9/6242

(57) ABSTRACT

A data processing device that makes effective use of a data group containing missing data is provided. A series of learning data containing missing data is acquired, and a representative value of data and a validity ratio representing a proportion of valid data being present are calculated from the series of learning data according to a predefined unit of aggregation. Then, learning of an estimation model is performed so as to minimize an error which is based on a difference between an output resulting from inputting the representative value and the validity ratio to the estimation model, and the representative value. Also, a series of estimation data containing missing data is acquired, and a representative value of data and a validity ratio representing a proportion of valid data being present are calculated from the series of estimation data according to a predefined unit of aggregation. Then, the representative value and the validity ratio are input to the learned estimation model and a feature value is acquired or data estimation is performed for the series of estimation data.

### TECHNICAL FIELD

An aspect of the present invention relates to a data processing device, a data processing method, and a program for making effective use of data containing missing data.

### BACKGROUND ART

With advancements in IoT (Internet of Things) technologies, an environment where household electronics such as a hemadynamometers and bathroom scales are connected to a network and health data such as blood pressure and body weight measured in daily life are collected through the network is being formed. Health data is often supposed to be measured regularly and in many cases contains information representing dates and times of measurement along with measured values. One issue with health data is that data is easily missing due to forgetting to measure, a fault of a measurement device and the like. Such missing data can lead to lower accuracy and the like in analysis of health data.

For data analysis with missing data taken into account, a learning method has been proposed and it takes the effect of missing data into consideration by using an array representing missing data to minimize an error only in portions without missing data (see Patent Literature 1, for example).

### CITATION LIST

### Patent Literature

Patent Literature 1: International Publication No. WO 2018/047655

### SUMMARY OF THE INVENTION

### Technical Problem

However, one possible issue with the analysis of data containing missing data is that data is reduced in amount. Particularly when an overall size of acquired data is small or when the proportion of missing data is large relative to the overall size of data, analysis disregarding missing data can result in a small amount of valid data.

For example, for health data that is measured multiples times a day like blood pressure, some of the measured values for a day can be missing. FIG. 4 shows an example of blood pressure measurement data for five days containing such data missing. In the example of FIG. 4, given a setting to measure the blood pressure three times a day, data with no missing data was obtained on June 22 and 26, while data for the second and third measurements is missing on the 23rd, data for the third measurement is missing on the 24th, and data for all the measurements is missing on the 25th. In such a case, if one decides to disregard data from days that have missing data even just once, for example, only data for two days could be used for analysis as valid data out of the data for five days.

Another issue is that degree of missingness is not taken into account. In the case of FIG. 4, for example, there is a difference in the degree of missingness, i.e., from the day with only one measurement missing to the day with all the three measurements missing. If determination is made only from the presence or absence of missing data, however, these days would all be determined to have missing data. As a unit of aggregation is larger, it can be more important to represent not only the presence or absence of missing data but the degree of missingness in an appropriate manner.

The present invention has been made in view of such circumstances and an object thereof is to provide a data processing device, a data processing method and a program for making effective use of data containing missing data.

### Means for Solving the Problem

To solve the above issues, a first aspect of the present invention provides a data processing device including: a data acquisition section that acquires a series of data containing missing data; a statistics calculation section that calculates a representative value of data and a validity ratio which represents a proportion of valid data being present from the series of data according to a predefined unit of aggregation; and a learning section that performs learning of an estimation model so as to minimize an error which is based on a difference between an output resulting from inputting the representative value and the validity ratio to the estimation model, and the representative value.

According to a second aspect of the present invention, in the first aspect, the learning section inputs to the estimation model an input vector made up of elements which are a concatenation of a predefined number of representative values and validity ratios corresponding to the respective representative values.

According to a third aspect of the present invention, in the second aspect, when X is defined as a vector with elements being the predefined number of representative values, W is defined as a vector with elements being validity ratios corresponding to the respective elements of X, and Y is defined as an output vector resulting from inputting the input vector to the estimation model, the learning section performs the learning of the estimation model so as to minimize an error L represented by L=|W·(Y−X)|².

According to a fourth aspect of the present invention, the first aspect further includes a first estimation section that, when a series of data containing missing data to be subjected to estimation is acquired by the data acquisition section, inputs representative values of the data and validity ratios representing the proportion of valid data being present calculated from the series of data by the statistics calculation section according to the unit of aggregation to the learned estimation model, and outputs an output from intermediate layers of the estimation model in response to the input as a feature value for the series of data.

According to a fifth aspect of the present invention, the first aspect further includes a second estimation section that, when a series of data containing missing data to be subjected to estimation is acquired by the data acquisition section, inputs representative values of the data and validity ratios representing the proportion of valid data being present calculated from the series of data by the statistics calculation section according to the unit of aggregation to the learned estimation model, and outputs an output from the estimation model in response to the input as estimated data with the missing data interpolated.

### Effects of the Invention

According to the first aspect of the present invention, a representative value of data and a validity ratio which represents the proportion of valid data being present are calculated from a series of data containing missing data according to a predefined unit of aggregation, and the estimation model is learned so as to minimize an error which is based on a difference between an output values resulting from inputting input values based on the representative value and the validity ratio to the estimation model, and the representative value.

As a result, even if the acquired series of data contains missing data, all the data can be effectively utilized as information per unit of aggregation without discarding data by calculating representative values and validity ratios as statistics according to a predefined unit of aggregation and using them for learning. Also, because not only whether there is missing data or not but the proportion of valid data being present in each unit of aggregation are calculated and used for learning, effective learning that takes into account even the degree of missingness can be performed.

According to the second aspect of the present invention, an input vector made up of elements which are a concatenation of a predefined number of representative values and validity ratios corresponding to the respective representative values is input to the estimation model and used for the learning of the estimation model. This enables learning to be performed with reliable association between the representative value and the validity ratio for each unit of aggregation without requiring complicated data processing even in a case where a learning data group contains missing data without regularity.

According to the third aspect of the present invention, learning of the estimation model is performed so as to minimize the error L=|W·(Y−X)|², which is calculated from the vector X with elements being a predefined number of representative values, the vector W with elements being validity ratios corresponding to the respective elements of X, and the vector Y resulting from inputting the input vector to the estimation model. As a result, the validity ratio is applied to both the input-side vector X and the output-side vector Y, enabling learning of the estimation model to be performed using an error that explicitly takes into account the degree of missingness.

According to the fourth aspect of the present invention, when a series of data containing missing data to be subjected to estimation is acquired, representative values of the data and validity ratios representing the proportion of valid data being present which are calculated from the series of data according to the unit of aggregation are input to the learned estimation model, and an output from intermediate layers of the estimation model in response to the input is output as a feature value for the series of data. This can provide a feature value that take into account even the degree of missingness for a series of data containing missing data, allowing a more accurate grasp of the features of the series of data.

According to the fifth aspect of the present invention, when a series of data containing missing data to be subjected to estimation is acquired, representative values of the data and validity ratios representing the proportion of valid data being present which are calculated from the series of data according to the unit of aggregation are input to the learned estimation model, and an output from the estimation model in response to the input is output as estimated data with the missing data interpolated. This can provide an estimation result that take into account even the degree of missingness for a series of data containing missing data.

Accordingly, the aspects of the present invention can provide techniques for making effective use of data containing missing data.

### BRIEF DESCRIPTION OF DRAWINGS

FIG. 1 is a block diagram showing a functional configuration of a data processing device according to an embodiment of the present invention.

FIG. 2 is a flowchart illustrating an example of a processing procedures and actions thereof in a learning phase performed by the data processing device shown in FIG. 1.

FIG. 3 is a flowchart illustrating an example of a processing procedures and actions thereof in an estimation phase performed by the data processing device shown in FIG. 1.

FIG. 4 shows an example of data containing missing data.

FIG. 5 shows an example of results of calculating statistics with a unit of aggregation of per day from data containing missing data.

FIG. 6 shows an example of an estimation model and an input and output to/from it.

FIG. 7 shows an example of results of calculating statistics with a unit of aggregation of per three days from data containing missing data.

FIG. 8 shows a first example of input vector generation.

FIG. 9 shows a second example of input vector generation.

FIG. 10 shows a first example of input vector generation based on multiple types of data.

FIG. 11 shows a second example of input vector generation based on multiple types of data.

### DESCRIPTION OF EMBODIMENTS

In the following, embodiments of the present invention are described with reference to the drawings.

### One Embodiment

(Configuration)

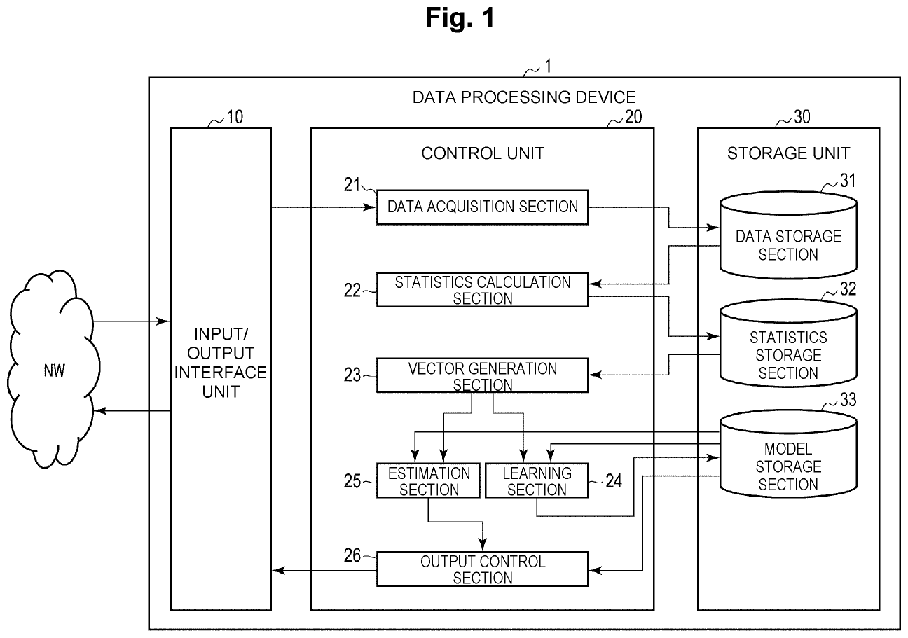

FIG. 1 is a block diagram showing a functional configuration of a data processing device 1 according to an embodiment of the present invention.

The data processing device 1 is managed by a medical institute, a healthcare center and the like, for example, and is composed of a server computer or a personal computer, for example. The data processing device 1 can acquire a series of data (also referred to as a “data group”) containing missing data, such as health data, via a network NW or via an input device not shown in the figure. The data processing device 1 may be installed on a stand-alone basis or may be provided as one of expansions to a terminal of a medical professional such as a doctor, an Electronic Medical Records (EMR) server installed in an individual medical institute, an Electronic Health Records (EHR) server installed in an individual region including multiple medical institutes, or even a cloud server of a service provider. Furthermore, the data processing device 1 may be provided as one of expansions to a user terminal and the like possessed by a user.

The data processing device 1 according to an embodiment includes an input/output interface unit 10, a control unit 20, and a storage unit 30.

The input/output interface unit 10 includes one or more wired or wireless communication interface units, for example, enabling transmission and reception of information to/from external apparatuses. The wired interface can be a wired LAN, for example, and the wireless interface can be an interface that supports low-power wireless data communication standards such as wireless LAN and Bluetooth (a registered trademark), for example.

For example, the input/output interface unit 10 performs processing for receiving data transmitted from a measurement device such as a hemadynamometer having communication capability, or accessing a database server to read stored data, and passing the data to the control unit 20 for analysis, under control of the control unit 20. The input/output interface unit 10 can also perform processing for outputting instruction information entered via an input device (not shown), such as a keyboard, to the control unit 20. The input/output interface unit 10 can further perform processing for outputting results of learning or results of estimation output from the control unit 20 to a display device (not shown) such as a liquid crystal display, or transmitting it to external apparatuses over the network NW.

The storage unit 30 uses non-volatile memory capable of dynamic writing and reading, e.g., a HDD (Hard Disk Drive) or an SSD (Solid State Drive), as a storage medium, and includes a data storage section 31, a statistics storage section 32, a model storage section 33 in addition to a program storage section as storage areas necessary for implementing this embodiment.

The data storage section 31 is used to store a data group for analysis acquired via the input/output interface unit 10.

The statistics storage section 32 is used to store statistics calculated from the data group.

The model storage section 33 is used to store an estimation model for estimating a data group with missing data in it interpolated from a data group containing missing data.

The storage sections 31 to 33 however are not essential components; the data processing device 1 may acquire necessary data from a measurement device or a user device when necessary. Alternatively, the storage sections 31 to 33 may not be built in the data processing device 1, but may be provided in an external storage medium such as a USB memory or a storage device such as a database server located in a cloud, for example.

The control unit 20 has a hardware processor such as a CPU (Central Processing Unit) and an MPU (Micro Processing Unit), not shown in the figure, and memories such as DRAM (Dynamic Random Access Memory) and SRAM (Static Random Access Memory), and includes a data acquisition section 21, a statistics calculation section 22, a vector generation section 23, a learning section 24, an estimation section 25, and an output control section 26 as processing functions necessary for implementing this embodiment. These processing functions are all implemented by execution of programs stored in the storage unit 30 by the processor. The control unit 20 may also be implemented in any of other various forms, including an integrated circuit such as ASIC (Application Specific Integrated Circuit) and FPGA (field-programmable gate array).

The data acquisition section 21 performs processing for acquiring a data group for analysis via the input/output interface unit 10 and storing them in the data storage section 31.

The statistics calculation section 22 performs processing for reading data stored in the data storage section 31, calculating statistics according to a predefined unit of aggregation, and storing the result of calculation in the statistics storage section 32. In an embodiment, statistics include a representative value of data included in each unit of aggregation and a validity ratio representing the proportion of valid data included in each unit of aggregation.

The vector generation section 23 performs processing for reading statistics stored in the statistics storage section 32 and generating a vector made up of a predefined number of elements. In an embodiment, the vector generation section 23 generates a vector X with elements being a predefined number of representative values, and a vector W with elements being validity ratios corresponding to the respective elements of the vector X. The vector generation section 23 outputs the generated vector X and vector W to the learning section 24 in a learning phase and to the estimation section 25 in an estimation phase.

The learning section 24 performs, in the learning phase, processing for reading the estimation model stored in the model storage section 33 and inputting the vector X and vector W received from the vector generation section 23 into the estimation model for learning of parameters of the estimation model. In an embodiment, the learning section 24 inputs a vector formed from a concatenation of the elements of the vector X and the elements of the vector W to the estimation model, and acquires a vector Y which is output by the estimation model in response to the input. Then, the learning section 24 performs processing for learning the parameters of the estimation model so as to minimize an error which is calculated based on a difference between the vector X and the vector Y and updating the estimation model stored in the model storage section 33 as necessary.

The estimation section 25 reads, in the estimation phase, the learned estimation model stored in the model storage section 33 and inputs the vector X and vector W received from the vector generation section 23 into the estimation model to perform data estimation processing. In an embodiment, the estimation section 25 inputs a vector formed from a concatenation of the elements of the vector X and the elements of the vector W to the learned estimation model, and outputs the vector Y or a feature value Z of intermediate layers which is output by the estimation model in response to the input to the output control section 26 as an estimation result.

The output control section 26 performs processing for outputting the vector Y or the feature value Z output by the estimation section 25. Alternatively, the output control section 26 can output parameters related to the learned estimation model stored in the model storage section 33.

(Operation)

Next, information processing operations of the data processing device 1 configured as described above are described. The data processing device 1 can accept an instruction signal from an operator entered such as through an input device and operate as the learning phase or the estimation phase, for example.

(1) Learning Phase

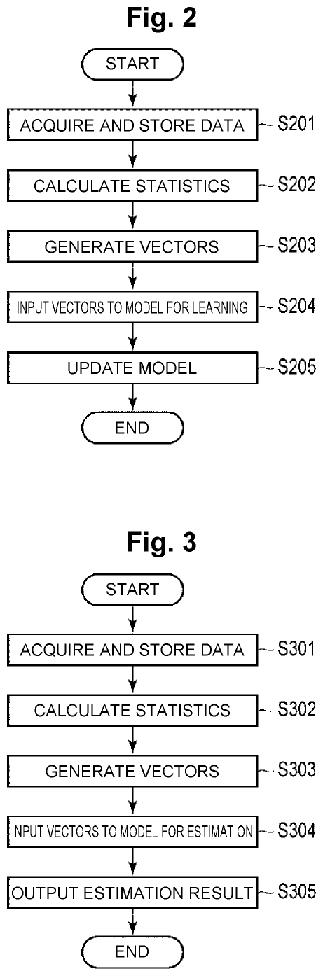

When the learning phase is set, the data processing device 1 executes learning processing for the estimation model as follows. FIG. 2 is a flowchart showing the procedure and actions of processing in the learning phase performed by the data processing device 1.

(1-1) Acquisition of Learning Data

First, at step S201, the data processing device 1 acquires a series of data containing missing data as learning data via the input/output interface unit 10, and stores the acquired data in the data storage section 31 under control of the data acquisition section 21.

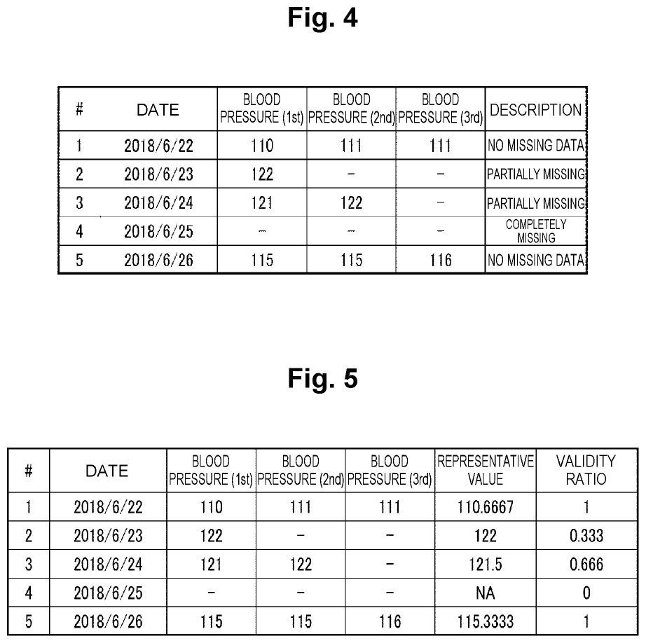

FIG. 4 illustrates measurement results for a particular user's blood pressure over five days with a set measurement frequency of three times a day, as an example of data that is acquired and stored. Three times a day may mean either measurements being made in different hours of day, e.g., immediately after awakening, before lunch and before going to bed, or measurements being repeated three times in the same hour of day. A blood pressure measurement may be any kind of measurement, such as systolic pressure, diastolic blood pressure, and pulse pressure. Also, the numerical values shown in FIG. 4 are shown merely for the purpose of illustration and are not meant to represent a particular health condition.

In addition, acquired data can also include a user ID, a device ID, information representing measurement dates/times and the like along with numerical data representing blood pressure measurements.

In FIG. 4, each record for one day is given a sequential number and annotated with description about missing data for the sake of convenience. In FIG. 4, the symbol “-” means that valid data is not present or data is missing. As shown in FIG. 4, on June 22 (#1) and 26 (#5), data was measured three times without missing data, whereas data was measured only once on the 23rd (#2), only twice on the 24th (#3), and data was not measured at all on the 25th (#4).

(1-2) Calculation of Statistics

Next, at step S202, the data processing device 1 performs processing for reading data stored in the data storage section 31 and calculating statistics according to a preset unit of aggregation under control of the statistics calculation section 22. The unit of aggregation is assumed to be set by the operator, designer, administrator and the like of the data processing device 1 as desired according to data type, for example, and stored in the storage unit 30. The statistics calculation section 22 reads the setting for the unit of aggregation stored in the storage unit 30, divides the data read from the data storage section 31 according to the unit of aggregation, and calculates statistics.

FIG. 5 shows representative values and validity ratios as statistics calculated with the data shown in FIG. 4. Here, the unit of aggregation is set to per day and the representative value is set to an average. However, the representative value is not limited thereto; a desired statistic such as median, maximum, minimum, mode, variance and standard deviation can be used. As with the unit of aggregation, the type of statistics to be calculated can also be preset by the administrator and the like.

In the example shown in FIG. 5, an average of valid data within each unit of aggregation is calculated as the representative value. For example, since blood pressure measurement data for three measurements (110, 111, 111) was obtained on June 22 (#1), “110.6667” (=(110+111+111)/3) has been calculated as the representative value (average). By contrast, on July 23 (#2), only blood pressure measurement data for one measurement (122) was obtained, so the representative value “122” (=122/1) has been calculated as an average of valid data. On June 25 (#4), measurement data was not obtained at all, so “NA” meaning being uncomputable is indicated.

The validity ratio indicates the proportion of valid data being present in the unit of aggregation. As shown in FIG. 5, when the unit of aggregation is one day and a measurement frequency of three times a day is set, the validity ratio will be calculated as “1(=3/3)” if measurement data for three measurements is obtained, as “0.666 (=2/3)” for two measurements, as “0.333 (=1/3)” for one measurement, and as “0 (=0/3)” for zero measurement.

The results thus calculated by the statistics calculation section 22 can be stored in the statistics storage section 32 as statistics data in association with identification numbers identifying the units of aggregation and/or date information, for example.

The unit of aggregation is not limited to per day but any unit can be employed. For example, it may be set to a certain time width such as per several hours, per three days and per week, or it may be a unit defined by the number of data containing missing data without using time information. Furthermore, units of aggregation may overlap each other. For example, they may be set such that with respect to a particular date, a moving average is calculated from data corresponding to two days, or the day before that date and the date in question.

(1-3) Generation of Vectors

Next, at step S203, the data processing device 1 performs processing for reading the statistics data stored in the statistics storage section 32 and generating two types of vectors (the vector X and the vector W) for use in the learning of the estimation model under control of the vector generation section 23.

The vector generation section 23 selects a preset number (n) of units of aggregation from the statistics data that has been read, extracts the representative values and validity ratios from the respective ones of the n units of aggregation, and generates the vector X (x₁, x₂, . . . , xₙ) with elements being n representative values and the vector W (w₁, w₂, . . . , wₙ) with elements being n validity ratios corresponding to the respective elements of the vector X. The number n of element corresponds to ½ of the number of input dimensions of the estimation model to be learned as mentioned later, and the number of input dimensions of the estimation model can be set as desired by the designer, administrator and the like of the data processing device 1. The number N of vector pairs (vector X and vector W) to be generated corresponds to the number of samples of learning data and this number N can also be set as desired.

For example, where the number of elements is set as n=3 and the number of vector pairs is set as N=2 in the example shown in FIG. 5, the vector generation section 23 can select the units of aggregation #1 to #3, for example, extract the representative values to generate a vector X₁ (110.6667, 122, 121.5), and extract the validity ratios to generate a vector W₁ (1, 0.333, 0.666) as the first vector pair. The vector generation section 23 can further select the units of aggregation #2 to #4, for example, and generate a vector X₂ (122, 121.5, 0) and a vector W₂ (0.333, 0.666, 0) as the second vector pair. As can be seen, the representative value “NA” can be replaced with 0 during the generation of vectors. Also as can be seen, the units of aggregation that are selected during the generation of vectors may or may not overlap each other. Instead of setting the number N of vector pairs to be generated, settings may be made so as to generate a number of vector pairs corresponding to all the combinations that can be selected from the statistics data that has been read.

The vector generation section 23 outputs the vector pair (the vector X and the vector W) generated in the above-described manner to the learning section 24.

(1-4) Learning of Estimation Model

Next, at step S204, the data processing device 1 reads the estimation model to be learned which is previously stored in the model storage section 33 and inputs the vector X and vector W received from the vector generation section 23 to the estimation model to perform the learning of it under control of the learning section 24. The estimation model to be learned can be set as desired by the designer, the administer or the like.

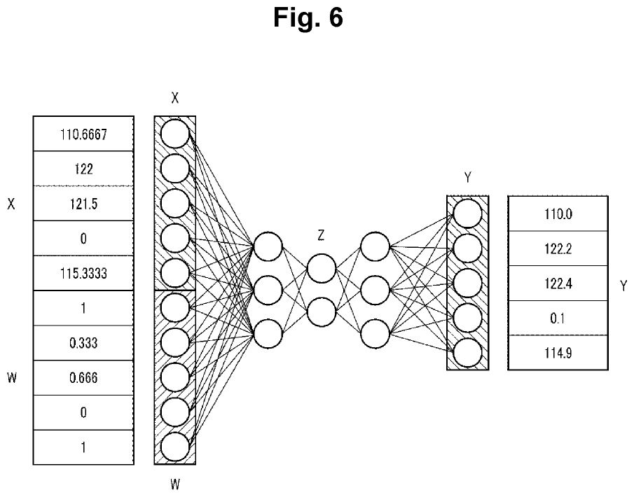

In an embodiment, a hierarchical neural network is used as the estimation model. FIG. 6 shows an example of such a neural network and a concept of input and output vectors for it. The estimation model shown in FIG. 6 consists of an input layer, three intermediate layers and an output layer, where the number of units in them are set as 10, 3, 2, 3, 5 in this order, respectively. However, these specific numbers of units are set only for the convenience of explanation and they may be set as desired depending on the nature of data to be analyzed, the purpose of analysis, working environment, etc. Also, the number of intermediate layers is not limited to three but any number of layers other than three can be selected to form the intermediate layers.

In a neural network, generally the elements of an input vector are input to the nodes of the input layer, in which they are weight-added and given a bias, then enter the nodes of the next layer, in which they are subjected to application of an activation function in that node and then output. Accordingly, where a weight coefficient is A, the bias is B, and the activation function is f, an output Q of an intermediate layer (a first layer) when P is input to the input layer is generally represented by:

Q=f(AP+B) (1).

In this embodiment, a vector formed from a concatenation of the elements of the vector X and the elements of the vector W is input to the input layer. In the example shown in FIG. 6, the vector X (110.6667, 122, 121.5, 0, 115.3333) and the vector W (1, 0.333, 0.666, 0, 1) are generated with the number of elements n=5 from the data of FIG. 5, and an input vector formed from a concatenation of their elements (110.6667, 122, 121.5, 0, 115.3333, 1, 0.333, 0.666, 0, 1) is input to the estimation model.

In FIG. 6, Y represents the output vector from the estimation model and has the same number of elements as the vector X. Therefore, in this embodiment, as the vector X and the vector W have the same number of elements, the number of output dimensions of the estimation model is ½ of the number of input dimensions. Also in the example of FIG. 6, the estimation model is designed such that the intermediate layers have a smaller number of units than in the input layer and the output layer.

In FIG. 6, Z represents the feature value of an intermediate layer. The feature value Z is obtained as the output from the nodes of the intermediate layer and can be represented based on Formula (1) above. For example, in the example of FIG. 6, a feature value Z₁ of an intermediate layer (a first layer) is represented by:

Z₁=f₁(A₁P+B₁) (2),

and a feature value Z₂ of an intermediate layer (a second layer) is represented by:

Z₂=f₂(A₂(f₁(A₁P+B₁))+B₂) (3).

The subscripts 1 and 2 mean that it is a parameter contributing the output of the first and the second layer, respectively.

A feature value generally represents what kind of features the input data has. It is known that a feature value Z that is obtained from a learned model in which the number of units in the intermediate layers are smaller than those in the input layer as shown in FIG. 6 can be beneficial information representing the intrinsic features of the input data with less dimensions.

The learning section 24 inputs an input vector formed from a concatenation of the elements of the vector X and the elements of the vector W to such an estimation model as discussed above, and acquires the output vector Y which is output by the estimation model in response to the input. Then, the learning section 24 performs learning of the parameters of the estimation model (such as the weight coefficient and the bias) so as to minimize an error L, calculated with Formula (4) below, for all the generated vector pairs (vector X and vector W).

L=|W·(Y−X)|² (4)

In Formula (4), it can be seen that the validity ratio vector W is applied to both the input-side vector X and the output-side vector Y, taking into account the degree of missingness in data in the learning of the estimation model.

In this manner, in the learning section 24, the estimation model is learned as an auto encoder so that the output from the output layer reproduces the input as much as possible. Here, the learning section 24 can perform learning of the estimation model so as to minimize the error L using stochastic gradient descent, such as Adam and AdaDelta, but these are not limiting and any other techniques can be used.

(1-5) Updating of the Model

After the parameters of the estimation model are determined so as to minimize the error L, the learning section 24 performs processing for updating the estimation model stored in the model storage section 33 at step S205. The data processing device 1 may also be configured to output the parameters of the learned model stored in the model storage section 33 through the output control section 26 in response to input of an instruction signal from the operator, for example, under control of the control unit 20.

When the learning phase ends, the data processing device 1 now can perform data estimation based on the newly acquired data group containing missing data using the learned model stored in the model storage section 33.

(2) Estimation Phase

When the estimation phase is set, the data processing device 1 can perform data estimation processing with the learned model as follows. FIG. 3 is a flowchart showing the procedure and actions of processing in the estimation phase performed by the data processing device 1. For processing similar to those in FIG. 2, detailed descriptions are omitted.

(2-1) Acquisition of Estimation Data

First, at step S301, the data processing device 1 acquires a series of data containing missing data as estimation data via the input/output interface unit 10 and stores the acquired data in the data storage section 31 as with step S201 under control of the data acquisition section 21.

(2-2) Calculation of Statistics

Next, at step S302, the data processing device 1 performs processing for reading the data stored in the data storage section 31 and calculating statistics according to a preset unit of aggregation as with step S202 under control of the statistics calculation section 22. For the unit of aggregation, the same settings as those used in the learning phase are preferably used; however, it is not necessarily limited to this. Likewise, for the representative value, the same representative value as that used in the learning phase (e.g., in the example above, an average of valid data) is preferably used; however, it is not necessarily limited to this. After the representative values and validity ratios have been calculated as statistics according to the unit of aggregation, the statistics calculation section 22 can store the results of calculation in the statistics storage section 32 as statistics data in association with identification numbers identifying the units of aggregation and/or date information, for example.

(2-3) Generation of Vectors

Next, at step S303, the data processing device 1 performs processing for reading the statistics data stored in the statistics storage section 32 and generating two types of vectors (the vector X and the vector W) for performing estimation as with step S203 under control of the vector generation section 23.

The vector generation section 23 selects a set number (n) of units of aggregation from the statistics data that has been read, extracts the representative values and validity ratios from the respective ones of the n units of aggregation, and generates the vector X (x if x₂, . . . , xₙ) with elements being n representative values and the vector W (w₁, w₂, . . . , wₙ) with elements being n validity ratios corresponding to the respective elements of the vector X. The number n of elements may be a stored value of n used in learning or may be obtained as the number of input dimensions of the learned model stored in the model storage section 33 multiplied by ½, for example.

The vector generation section 23 outputs the generated vector pair (the vector X and the vector W) to the estimation section 25.

(2-4) Data Estimation

Next, at step S304, the data processing device 1 performs processing for reading the learned estimation model stored in the model storage section 33, and inputting the vector X and vector W received from the vector generation section 23 to the learned estimation model to acquire the output vector Y which is output by the estimation model in response to the input, under control of the estimation section 25. As described in the learning phase, the output vector Y shown in FIG. 6 is represented by:

Y=f₄(A₄(f₃(A₃(f₂(A₂(f₁(A₁P+B₁))+B₂))+B₃))+B₄) (5)

In the example shown in FIG. 6, the output vector Y (110.0, 122.2, 122.4, 0.1, 114.9) is output from the estimation model. The elements of the input vector X have been replaced with numerical values that take into account validity ratios in the vector Y, in particular, x₄=0 (missing data) in the vector X has been replaced with y₄=0.1 in the vector Y.

(2-5) Outputting of Estimation Result

At step S305, the data processing device 1 can output the result of estimation by the estimation section 25 via the input/output interface unit 10 in response to input of an instruction signal from the operator, for example, under control of the output control section 26. The output control section 26 can take the output vector Y output from the estimation model and output it on a display device such as a liquid crystal display or transmit it to external apparatuses over the network NW as a data group having missing data corresponding to the input data group interpolated, for example.

Alternatively, the output control section 26 can extract and output the feature value Z of the intermediate layers corresponding to the input data group. The feature value Z can be considered to be representing the intrinsic features of the input data group with less dimensions than the original input data group as noted above. Therefore, use of the feature value Z as the input to a certain separate learner enables processing with reduced load compared to when the original input data group is directly used. For such a separate learner, application to a classifier such as logistic regression, support vector machine and random forest, or a regression model using multiple regression analysis or regression tree is conceivable, for example.

(Effects)

As detailed above, in an embodiment of the present invention, a series of data containing missing data are acquired by the data acquisition section 21, and from this series of data, representative values of the data and validity ratios representing the proportion of valid data being present are calculated as statistics according to a predetermined unit of aggregation by the statistics calculation section 22. In the calculation of the validity ratio, missing data is represented by a continuous value as a proportion in the embodiment above, rather than being represented by binary values of present/absent.

Then, in the learning phase, the vector X with elements being representative values extracted from a predetermined number n of units of aggregation and the vector W with elements being the corresponding validity ratios are generated by the vector generation section 23. Next, an input vector formed from a concatenation of the elements of the vector X and the elements of the vector W is input to the estimation model by the learning section 24, and learning of the estimation model is performed as an auto encoder so as to minimize the error L, which is based on the vector Y output by the estimation model in response to the input.

As a result, even when some data or all the data in a unit of aggregation are missing, the data can be effectively utilized for use in learning without discarding the unit of aggregation so that reduction in data can be prevented in the learning of the estimation model. This is particularly advantageous when the proportion of missing data is large relative to the overall size of data or when the overall size of data is small.

Further, according to the embodiment above, for the representative values in the respective units of aggregation, learning can be performed taking into account the degree of missingness for the respective units of aggregation. Since learning is performed so that data with larger missing data contributes less by way of the W contained in the error L as shown in Formula (4), even the degree of missingness can be effectively employed to make effective use of data.

Also in the estimation phase, the vector X with elements being representative values extracted from a predetermined number n of units of aggregation and the vector W with elements being the corresponding validity ratios are generated by the vector generation section 23 as in the learning phase. Then, an input vector formed from a concatenation of the elements of the vector X and the elements of the vector W is input by the estimation section 25 to the learned estimation model which has been learned as described above, and the vector Y which is output by the estimation model in response to the input or the feature value Z which is output from the intermediate layers is acquired.

Thus, estimation processing can be performed with effective use of the original data without discarding data and also in consideration of even the degree of missingness when data is estimated using a learned estimation model on the basis of a data group containing missing data or when feature values are obtained from the intermediate layers of the learned estimation model.

Furthermore, since the embodiment above does not require excessively complicated manipulations for calculating statistics or generating the input vector for either the learning phase or the estimation phase, it can be implemented with desired settings or modifications by the administrator or the like according to the nature of data or the purpose of analysis.

### OTHER EMBODIMENTS

The present invention is not limited to the foregoing embodiments.

For example, while in relation to FIGS. 5 and 6 the vector generation section 23 was described as extracting representative values and validity ratios calculated according to the unit of aggregation as many as a predetermined number of elements and generating the vector X and the vector W, the vector X may be generated from raw data prior to calculation of statistics.

For instance, in the example of FIG. 4, a vector X₁ (110, 111, 111) can also be generated by directly extracting measured values from the record for #1. In this case, as the corresponding vector W₁, a vector W₁ (1, 1, 1) can be generated by using “1” as the validity ratio because the record of #1 has no missing data, for example. Similarly, a vector X₂ (122, 0, 0) can be generated from the record of #2 in FIG. 4. In this case, as the corresponding vector W₂, a vector W₂ (0.333, 0.333, 0.333) can be generated using “0.333” as the validity ratio because only the measured value for the first measurement was obtained in the record of #2. Alternatively, a vector W₂ (1, 0, 0) may be generated assuming that only the measure value for the first measurement was valid.

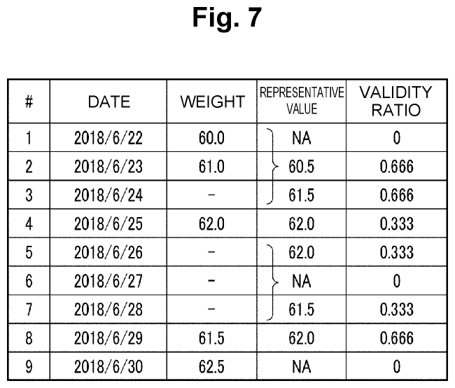

The unit of aggregation that is used by the statistics calculation section 22 is not limited to the embodiment above but any unit of aggregation can be set. FIG. 7 shows an example of a way to calculate statistics when the unit of aggregation is three days. In FIG. 7, averages and validity ratios over consecutive three days as the unit of aggregation are calculated from measurement data representing body weights measured on individual days. That is, in FIG. 7, for #2 associated with June 23, an average (representative value) “60.5” over the three days from June 22 through 24 and the validity ratio (the proportion of valid data being present) “0.666” for the same three days have been calculated as statistics. Likewise, for #6 associated with June 27, “NA (uncomputable)” as the representative value and the validity ratio of “0” have been calculated because measurement data was not obtained at all on the three days from June 26 through 28. As previously mentioned, “NA” can be replaced with “0” during generation of vectors.

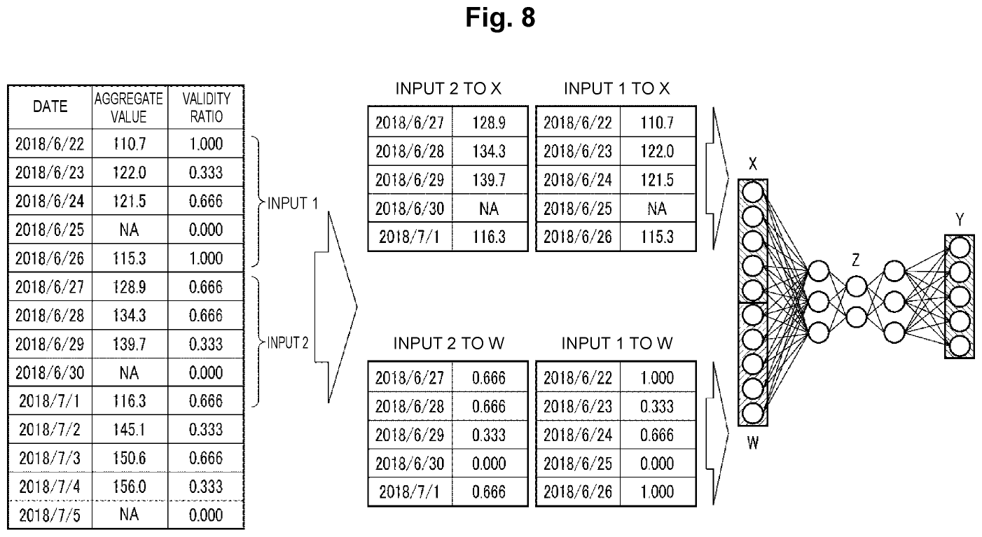

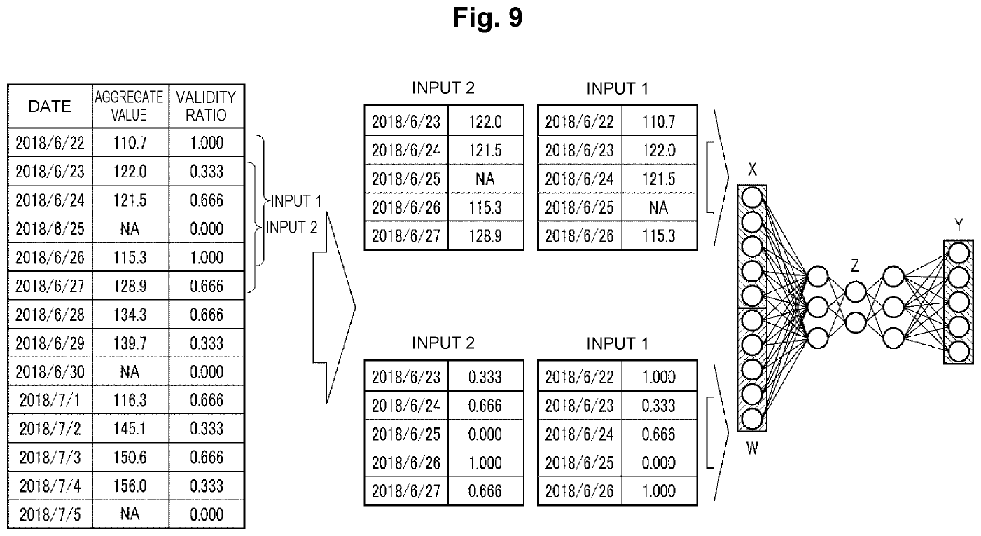

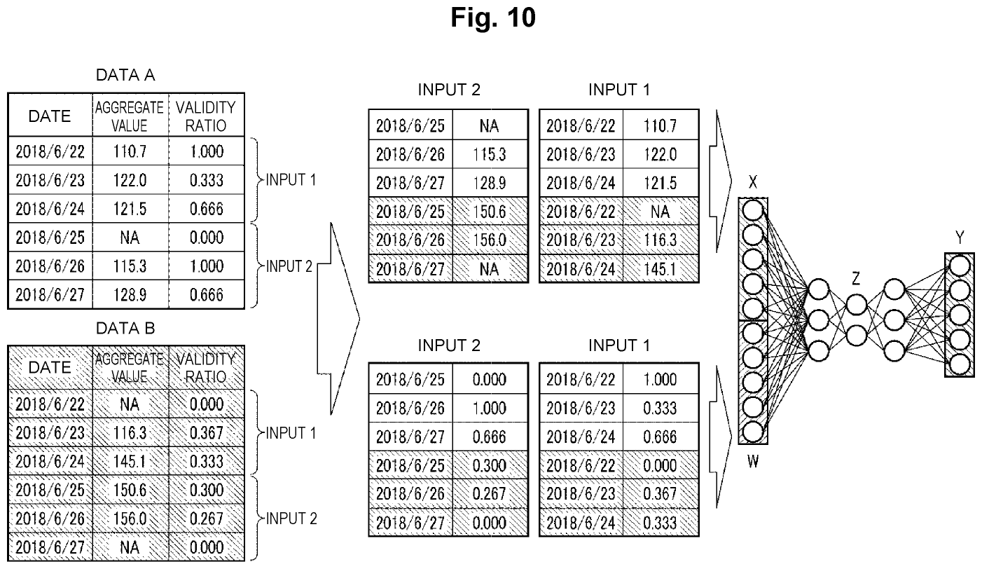

Moreover, generation of vectors by the vector generation section 23 is not limited to the above described embodiment. FIGS. 8 and 9 show an example of data extraction in five dimensions from time-series data for generating vectors. In the example of FIG. 8, the original data is divided into five-day blocks and input to an estimation model such as shown in FIG. 6. In the example of FIG. 9, data for five days is extracted while shifting by one day to form the input vector. Similarly, extraction with shifting by two, three or four days is also possible and any other method of extraction may be adopted and applied to the embodiment above.

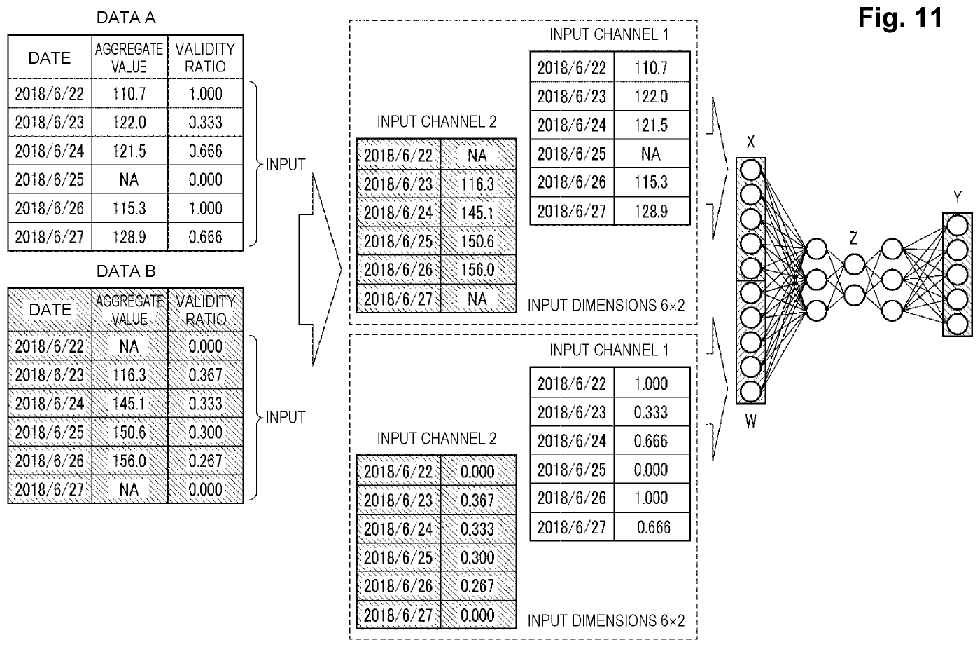

Furthermore, the embodiment above can be applied when multiple types of data are present. FIGS. 10 and 11 show an example of input vector generation from two types of data (data A and data B). In this example, “data A” is assumed to be health-related data such as blood pressure values and body weight, laboratory test results such as blood glucose level and urinalysis values, or answers to a history taking (a questionnaire), while “data B” is assumed to be sensor data as measured by a wearable device such as the number of steps taken or sleeping hours, position information measured by GPS and the like, or answers to a history taking (a questionnaire). For example, blood pressure measurement data can be collected as “data A” and step measurement data can be collected as “data B”, both of which are considered and analyzed at the same time so as to help a subject's health management or prevention of diseases. However, the embodiment above is not limited to such health-related data but various types of data that are obtained in various fields, such as manufacture, transportation, and agriculture.

When two types of data are present as shown in FIG. 10, an input vector can be generated by concatenating data extracted from each type of data. In the example of FIG. 10, for an input of six dimensions, the first three dimensions are assigned to data A and the last three dimensions are assigned to data B, and data for three days extracted from each of data A and data B are used as the input vector. While the example of FIG. 10 describes a case of extracting data with shifting by the same period as the input dimension, data may be input while shifting by one day as mentioned above in relation to FIG. 9. The example of FIG. 10 is also applicable to a case with more than two types of data.

Alternatively, as shown in FIG. 11, multiple data may be input so as to be assigned to input channels, respectively. This is implemented by a general method that is used to input image data to a neural network and the like when one pixel has three pieces of information, like an RGB image.

While the embodiment above was described taking time-series data that is recorded on a daily basis in particular as an example, the recording frequency of data does not have to be one day but data recorded at any frequency can be used.

Furthermore, the embodiment above can also be applied to data other than time-series data as noted above. For example, it can be temperature data recorded at different observation points or image data. For data that is represented by a two-dimensional array like image data, implementation is done by extracting data per row and concatenating and inputting them, as discussed for an instance where multiple types of data are present.

The embodiment above can also be applied to a compilation result for questionnaires or tests. For example, in the case of questionnaires, it is expected that data will be missing for some questions or data with completely no answers in relation to particular subjects will be obtained for reasons such as not applicable or unwillingness to answer. Even in such a situation, the embodiment above permits learning and estimation to be performed while distinguishing and taking into account partially unanswered and completely unanswered and making effective use of data without discarding it. Where data contains verbal information such as free answers to questionnaires, the embodiment above can be applied after converting data into numerical values by a certain method such as analyzing the frequency of appearance of keywords with text mining.

Moreover, all of the functional components of the data processing device 1 need not necessarily provided in a single device. For example, the functional components 21 to 26 of the data processing device 1 may be distributed across cloud computers, edge routers and the like such that those devices cooperate with each other to perform learning and estimation. This can reduce processing burden on the individual devices and improve processing efficiency.

Additionally, calculation of statistics, data storage format and the like can be implemented with various modifications within the scope of the present invention.

In short, the present invention is not limited to the exact embodiments described above but the components can be embodied with modifications within the scope of the invention in practicing stage. Also, different inventions can be formed by appropriate combination of the multiple components disclosed in the embodiment above. For example, several components may be removed from all the components shown in the embodiments. Further, components from different embodiments may be combined as appropriate.

### REFERENCE SIGNS LIST

1 data processing device
10 input/output interface unit
20 control unit
21 data acquisition section
22 statistics calculation section
23 vector generation section
24 learning section
25 estimation section
26 output control section
30 storage unit
31 data storage section
32 statistics storage section
33 model storage section

1. A data processing device comprising:
a data acquisition section, including one or more processors, configured to acquire a series of data containing missing data;
a statistics calculation section, including one or more processors, configured to calculate a representative value of data and a validity ratio which represents a proportion of valid data being present from the series of data according to a predefined unit of aggregation; and
a learning section, including one or more processors, configured to perform learning of an estimation model so as to minimize an error which is based on a difference between an output resulting from inputting the representative value and the validity ratio to the estimation model, and the representative value.

2. The data processing device according to claim 1, wherein
the learning section is configured to input to the estimation model an input vector made up of elements which are a concatenation of a predefined number of representative values and validity ratios corresponding to the respective representative values.

3. The data processing device according to claim 2, wherein
when X is defined as a vector with elements being the predefined number of representative values, W is defined as a vector with elements being validity ratios corresponding to the respective elements of X, and Y is defined as an output vector resulting from inputting the input vector to the estimation model,
the learning section is configured to perform the learning of the estimation model so as to minimize an error L represented by:
L=|W·(Y−X)|².

4. The data processing device according to claim 1, further comprising a first estimation section, including one or more processors, that, when a series of data containing missing data to be subjected to estimation is acquired by the data acquisition section, is configured to input representative values of the data and validity ratios representing the proportion of valid data being present calculated from the series of data by the statistics calculation section according to the predefined unit of aggregation to the learned estimation model, and output an output from intermediate layers of the estimation model in response to the input as a feature value for the series of data.

5. The data processing device according to claim 1, further comprising a second estimation section, including one or more processors, that, when a series of data containing missing data to be subjected to estimation is acquired by the data acquisition section, is configured to input representative values of the data and validity ratios representing the proportion of valid data being present calculated from the series of data by the statistics calculation section according to the predefined unit of aggregation to the learned estimation model, and output an output from the estimation model in response to the input as estimated data with the missing data interpolated.

6. A data processing method to be performed by a data processing device, the method comprising:
acquiring a series of data containing missing data;
calculating a representative value of data and a validity ratio which represents a proportion of valid data being present from the series of data according to a predefined unit of aggregation; and
performing learning of an estimation model so as to minimize an error which is based on a difference between an output resulting from inputting the representative value and the validity ratio to the estimation model, and the representative value.

7. The data processing method according to claim 6, wherein when X is defined as a vector with elements being a predefined number of representative values, W is defined as a vector with elements being validity ratios corresponding to the respective elements of X, and Y is defined as an output vector resulting from inputting an input vector made up of elements which are a concatenation of the elements of the vector X and the elements of the vector W to the estimation model,
performing learning of the estimation model comprises performing the learning of the estimation model so as to minimize an error L represented by:
L=|W·(Y−X)|².

8. A non-transitory computer readable medium storing one or more instructions for causing a processor to execute:
acquiring a series of data containing missing data;
calculating a representative value of data and a validity ratio which represents a proportion of valid data being present from the series of data according to a predefined unit of aggregation; and
performing learning of an estimation model so as to minimize an error which is based on a difference between an output resulting from inputting the representative value and the validity ratio to the estimation model, and the representative value.

9. The data processing method according to claim 6, further comprising:
when a series of data containing missing data to be subjected to estimation is acquired,
inputting representative values of the data and validity ratios representing the proportion of valid data being present calculated from the series of data according to the predefined unit of aggregation to the learned estimation model; and
outputting an output from intermediate layers of the estimation model in response to the input as a feature value for the series of data.

10. The data processing method according to claim 6, further comprising:
when a series of data containing missing data to be subjected to estimation is acquired,
inputting representative values of the data and validity ratios representing the proportion of valid data being present calculated from the series of data according to the predefined unit of aggregation to the learned estimation model; and
outputting an output from the estimation model in response to the input as estimated data with the missing data interpolated.

11. The non-transitory computer readable medium according to claim 8, wherein when X is defined as a vector with elements being a predefined number of representative values, W is defined as a vector with elements being validity ratios corresponding to the respective elements of X, and Y is defined as an output vector resulting from inputting an input vector made up of elements which are a concatenation of the elements of the vector X and the elements of the vector W to the estimation model,
performing learning of the estimation model comprises performing the learning of the estimation model so as to minimize an error L represented by:
L=|W·(Y−X)|².

12. The non-transitory computer readable medium according to claim 8, wherein the one or more instructions further cause the processor to execute:
when a series of data containing missing data to be subjected to estimation is acquired,
inputting representative values of the data and validity ratios representing the proportion of valid data being present calculated from the series of data according to the predefined unit of aggregation to the learned estimation model; and
outputting an output from intermediate layers of the estimation model in response to the input as a feature value for the series of data.

13. The non-transitory computer readable medium according to claim 8, wherein the one or more instructions further cause the processor to execute:
when a series of data containing missing data to be subjected to estimation is acquired,
inputting representative values of the data and validity ratios representing the proportion of valid data being present calculated from the series of data according to the predefined unit of aggregation to the learned estimation model; and
outputting an output from the estimation model in response to the input as estimated data with the missing data interpolated.
